SEQUENCE LISTING (10 sequences)

SEQ ID NO: 1 - DNA, 2730 bp, Corynebacterium glutamicum
  CDS (743)..(2026)
ctgtaagatagctcaacagttaaagtcagattgaccctaaggtggtttcttgcccgcttcacctgaaatccagatggccgtatccacgatcatcttcgcgctgcgccccggcccccaggatctccccagcctgtgggcccccttcgttccgcgcacccgcgaaccacatttaaataaatgggcactgcccggcggttggctgccaccacatgaagaacttgaagatgctgctgcccgcacactcgcagaaaccaccggcctgcaccccagctatctagaacagctctacactttcggaaaagtcgaccgctccccaaccggacgcgtgatctctgtggtgtattgggcacttgtccgagccgatgaagcgttgaaagccatcccaggagaaaacgtccagtggtttcccgccgatcatctccctgagctggcatttgaccacaataacatcgtcaaatatgcactagaacgacttcgcaccaaggtggaatactccgaaatcgcccactccttcctcggagaaaccttcaccatcgcccagcttcgatccgtgcatgaggcagtccttggacacaaactcgatgccgccaacttccgaagatccgtggccacctcgcccgatctgatcgacaccggcgaagtgcttgcgggaacaccgcaccgcccacccaaactgttcagattccaaagataaattctgacgctcattccagcccaccgtttagaagaaaagaccccaatcatgaccacctcaatcaccccatctgtcaaccttgcattgaaaaatgccaatagctgcaacagtgaactcaaagacggaccctggttcctcgaccagcccggaatgccggatgtctacggccccggcgcgtcacaaaacgatccgatccctgcgcatgctccgcgccagcaggttctccccgaggagtaccagcgcgcaagtgatgacgaactgcatcgtaggatccgggaagcgaaagacaccctgggtgacaaagtggttatcctaggacacttctaccagcgcgatgaagttatccaacacgcagattttgttggtgactctttccaacttgcccgcgctgccaaaacccgacccgaggcggaagcgattgtgttctgcggtgtgcacttcatggctgaaaccgctgatctgttatccacggatgaacaatcagtgatcctccccaaccttgccgcaggttgctccatggcagacatggctgaccttgattccgtcgaagactgctgggagcaactcacctcaatttatggcgatgacaccctgatccctgtgacctacatgaattcctctgcagcgctcaaaggtttcgtgggtgagcacggcggaattgtatgcacctcctcaaatgcacgttccgtattggagtgggcgtttgaacgcggccaacgagtcctgttcttccccgatcagcacttgggtcgaaacaccgcgaaagccatgggcattgggatcgatcaaatgcccctgtggaatcccaacaaaccactgggtggcaacaccgtttccgagctagaaaacgcaaaggtactgctctggcatggtttctgctctgtacacaagcgctttactgtcgagcagatcaacaaagcccgcgccgagtaccccgacgttcacgtcatcgtgcaccctgaatcccccatgccagttgttgacgccgccgactcatccggatccactgacttcattgtgaaagccattcaagcagcaccggcaggatctacctttgcgatcggcaccgaaatcaacttggttcagcgcctggcagcccagtacccgcagcacaccatcttctgcctcgaccctgtcatctgcccatgctccaccatgtatcgcattcaccctggttacctggcctgggcacttgaggagttggtggctggaaacgtgattaaccagatttctgtctctgaatccgtggcggcaccggcgcgagtcgctttggaaaggatgctatctgttgttccagcagctcctgttactcctagctcctcgaaggatgcgtaatttatgactacccatattgaccgcatcgttggcgcagcgttatccgaggatgcgccatggggcgacattacctccgacacttttatcccaggatcggcgcagctgagcgccaaggttgttgcccgggagccaggtgtgttcagcgggcaggcgcttttcgacgcctccttccggctcgtcgatcctaggataaacgcatcccttaaggtggctgatggtgacagctttgaaaccggggacatcctaggaacaattaccggcagtgctagaagcatcctccgttcagagcgcattgctctcaacttcattcagaggacgtccggcatcgctacattgacatcgtgctatgttgcagaggttaaaggcaccaaagcccgcattgttgatacccggaaaaccacacccggcctgcgcatcattgaacgccaagctgtccgtgacggtggcggatttaatcaccgagccaccttgtccgatgctgtcatggtgaaagataaccatctcgcagccatcgcatcccaggggctcagcatcactgaagcgctgtcgaatatgaaagctaaactcccccacaccacccatgtggaagtcgaagttgatcatatagagcagatcgaaccagttcttgctgctggtgtggacaccatcatgttggataatttcaccattgatcagctcatcgaaggcgttgatctcattgg

SEQ ID NO: 2 - protein, 428 aa, Corynebacterium glutamicum
MTTSITPSVNLALKNANSCNSELKDGPWFLDQPGMPDVYGPGASQNDPIPAHAPRQQVLPEEYQRASDDELHRRIREAKDTLGDKVVILGHFYQRDEVIQHADFVGDSFQLARAAKTRPEAEAIVFCGVHFMAETADLLSTDEQSVILPNLAAGCSMADMADLDSVEDCWEQLTSIYGDDTLIPVTYMNSSAALKGFVGEHGGIVCTSSNARSVLEWAFERGQRVLFFPDQHLGRNTAKAMGIGIDQMPLWNPNKPLGGNTVSELENAKVLLWHGFCSVHKRFTVEQINKARAEYPDVHVIVHPESPMPVVDAADSSGSTDFIVKAIQAAPAGSTFAIGTEINLVQRLAAQYPQHTIFCLDPVICPCSTMYRIHPGYLAWALEELVAGNVINQISVSESVAAPARVALERMLSVVPAAPVTPSSSKDA

SEQ ID NO: 3 - DNA, 2270 bp, Corynebacterium glutamicum
  CDS (701)..(1537)
cgtgggtgagcacggcggaattgtatgcacctcctcaaatgcacgttccgtattggagtgggcgtttgaacgcggccaacgagtcctgttcttccccgatcagcacttgggtcgaaacaccgcgaaagccatgggcattgggatcgatcaaatgcccctgtggaatcccaacaaaccactgggtggcaacaccgtttccgagctagaaaacgcaaaggtactgctctggcatggtttctgctctgtacacaagcgctttactgtcgagcagatcaacaaagcccgcgccgagtaccccgacgttcacgtcatcgtgcaccctgaatcccccatgccagttgttgacgccgccgactcatccggatccactgacttcattgtgaaagccattcaagcagcaccggcaggatctacctttgcgatcggcaccgaaatcaacttggttcagcgcctggcagcccagtacccgcagcacaccatcttctgcctcgaccctgtcatctgcccatgctccaccatgtatcgcattcaccctggttacctggcctgggcacttgaggagttggtggctggaaacgtgattaaccagatttctgtctctgaatccgtggcggcaccggcgcgagtcgctttggaaaggatgctatctgttgttccagcagctcctgttactcctagctcctcgaaggatgcgtaatttatgactacccatattgaccgcatcgttggcgcagcgttatccgaggatgcgccatggggcgacattacctccgacacttttatcccaggatcggcgcagctgagcgccaaggttgttgcccgggagccaggtgtgttcagcgggcaggcgcttttcgacgcctccttccggctcgtcgatcctaggataaacgcatcccttaaggtggctgatggtgacagctttgaaaccggggacatcctaggaacaattaccggcagtgctagaagcatcctccgttcagagcgcattgctctcaacttcattcagaggacgtccggcatcgctacattgacatcgtgctatgttgcagaggttaaaggcaccaaagcccgcattgttgatacccggaaaaccacacccggcctgcgcatcattgaacgccaagctgtccgtgacggtggcggatttaatcaccgagccaccttgtccgatgctgtcatggtgaaagataaccatctcgcagccatcgcatcccaggggctcagcatcactgaagcgctgtcgaatatgaaagctaaactcccccacaccacccatgtggaagtcgaagttgatcatatagagcagatcgaaccagttcttgctgctggtgtggacaccatcatgttggataatttcaccattgatcagctcatcgaaggcgttgatctcattggtggacgtgcactggtggaagcatctggcggagtcaacctcaacaccgcgggaaagattgcatcaaccggtgtcgacgtcatttccgttggagcgcttacccattctgtgcatgcacttgacctaggactcgatattttctaatgctctaccttgataatgcagccaccaccagtgtgcgcaatgaagcacttgaggccatgtggccttatctcaccggagcgtttggcaatccgtcaagtccccatgaggtgggaagactcgcctctgcggggctggaggatgctcgaactcgggtggcccgcattatcggaggacgccccacacaggtgacgtttacgtcgggtggatcagaagccaacaacctcgctatcaaaggagcgtgcttagctaatcctcgtggccggcacctcatcaccaccccgatcgagcatgacagtgtcctagaaactgctgcttatcttgaaaggtttcatgatttcgagatcacctacctatcccccgatcacactgggctgatctccccggagggtctccgcaaagcagtcaggccggacaccacattgatcagcattggttatgccaacaatgaggtgggaaccattcagccgatagctgagttggcggcggtaagcagtacgccttttcacaccgatgcagtgcaagctgcacatttaacctttgacttgggagttgacgcgttaagtttgtcgggtcataaattcggtgcgcctaaagggattggagtgttatggtcaaagcttcccctggagccggtaatccatggcggcggccaggaaaaagggcggcgtagtggcacggaaaacgttgcgggggctatcgcctttgccactgccttggaattggccagggc

SEQ ID NO: 4 - protein, 279 aa, Corynebacterium glutamicum
MTTHIDRIVGAALSEDAPWGDITSDTFIPGSAQLSAKVVAREPGVFSGQALFDASFRLVDPRINASLKVADGDSFETGDILGTITGSARSILRSERIALNFIQRTSGIATLTSCYVAEVKGTKARIVDTRKTTPGLRIIERQAVRDGGGFNHRATLSDAVMVKDNHLAAIASQGLSITEALSNMKAKLPHTTHVEVEVDHIEQIEPVLAAGVDTIMLDNFTIDQLIEGVDLIGGRALVEASGGVNLNTAGKIASTGVDVISVGALTHSVHALDLGLDIF

SEQ ID NO: 5 - DNA, 718 bp, Corynebacterium glutamicum
  primer_bind (1)..(20)
aagcgattgtgttctgcggtgtgcacttcatggctgaaaccgctgatctgttatccacggatgaacaatcagtgatcctccccaaccttgccgcaggttgctccatggcagacatggctgaccttgattccgtcgaagactgctgggagcaactcacctcaatttatggcgatgacaccctgatccctgtgacctacatgaattcctctgcagcgctcaaaggtttcgtgggtgagcacggcggaattgtatgcacctcctcaaatgcacgttccgtattggagtgggcgtttgaacgcggccaacgagtcctgttcttccccgatcagcacttgggtcgaaacaccgcgaaagccatgggcattgggatcgatcaaatgcccctgtggaatcccaacaaaccactgggtggcaacaccgtttccgagctagaaaacgcaaaggtactgctctggcatggtttctgctctgtacacaagcgctttactgtcgagcagatcaacaaagcccgcgccgagtaccccgacgttcacgtcatcgtgcaccctgaatcccccatgccagttgttgacgccgccgactcatccggatccactgacttcattgtgaaagccattcaagcagcaccggcaggatctacctttgcgatcggcaccgaaatcaacttggttcagcgcctggcagcccagtacccgcagcacaccatcttctgcctcga

SEQ ID NO: 6 - DNA, 582 bp, Corynebacterium glutamicum
  primer_bind (1)..(20)
agctgagcgccaaggttgttgcccgggagccaggtgtgttcagcgggcaggcgcttttcgacgcctccttccggctcgtcgatcctaggataaacgcatcccttaaggtggctgatggtgacagctttgaaaccggggacatcctaggaacaattaccggcagtgctagaagcatcctccgttcagagcgcattgctctcaacttcattcagaggacgtccggcatcgctacattgacatcgtgctatgttgcagaggttaaaggcaccaaagcccgcattgttgatacccggaaaaccacacccggcctgcgcatcattgaacgccaagctgtccgtgacggtggcggatttaatcaccgagccaccttgtccgatgctgtcatggtgaaagataaccatctcgcagccatcgcatcccaggggctcagcatcactgaagcgctgtcgaatatgaaagctaaactcccccacaccacccatgtggaagtcgaagttgatcatatagagcagatcgaaccagttcttgctgctggtgtggacaccatcatgttggataatttcaccattgatcagctcatcg

SEQ ID NO: 7 - DNA, 20 bp, Artificial Sequence
  Synthetic DNA
aagcgattgtgttctgcggt

SEQ ID NO: 8 - DNA, 20 bp, Artificial Sequence
  Synthetic DNA
tcgaggcagaagatggtgtg

SEQ ID NO: 9 - DNA, 20 bp, Artificial Sequence
  Synthetic DNA
agctgagcgccaaggttgtt

SEQ ID NO: 10 - DNA, 20 bp, Artificial Sequence
  Synthetic DNA
cgatgagctgatcaatggtg

(19) United States
(12) Patent Application Publication
(10) Pub. No.: US 2002/0168732 A1
(43) Pub. Date: Nov. 14, 2002

(54) Process for the fermentative preparation of L-amino acids using coryneform bacteria

(76) Inventors: Bettina Moeckel, Duesseldorf (DE); Thomas Hermann, Bielefeld (DE); Walter Pfefferle, Halle (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 09/816,079

(22) Filed: Mar. 26, 2001

(30) Foreign Application Priority Data
Nov. 10, 2000 (DE) 100 55 871.2
Mar. 3, 2001 (DE) 101 10 344.1

(51) Int. Cl. C12P 13/08; C12N 1/21

(52) U.S. Cl. 435/115; 435/252.3

(57) ABSTRACT

The invention relates to a process for the preparation of L-amino acids in which the following steps are carried out,

a) fermentation of the coryneform bacteria which produce the desired L-amino acid and in which at least the nadA and/or nadC gene is or are attenuated,

b) concentration of the desired L-amino acid in the medium or in the cells of the bacteria, and

c) isolation of the L-amino acid,

and optionally bacteria in which further genes of the biosynthesis pathway of the desired L-amino acid are additionally enhanced are employed, or bacteria in which the metabolic pathways which reduce the formation of the desired L-amino acid are at least partly eliminated are employed.

The invention relates to a process for the fermentative preparation of L-amino acids, in particular L-valine and L-lysine, using coryneform bacteria in which the nadA and/or nadC gene is or are attenuated.

### PRIOR ART

L-Amino acids, in particular L-valine and L-lysine, are used in human medicine and in the pharmaceuticals industry, in the foodstuffs industry and very particularly in animal nutrition.

It is known that amino acids are prepared by fermentation from strains of coryneform bacteria, in particular Corynebacterium glutamicum. Because of their great importance, work is constantly being undertaken to improve the preparation processes. Improvements to the process can relate to fermentation measures, such as, for example, stirring and supply of oxygen, or the composition of the nutrient media, such as, for example, the sugar concentration during the fermentation, or the working up to the product form by, for example, ion exchange chromatography, or the intrinsic output properties of the microorganism itself.

Methods of mutagenesis, selection and mutant selection are used to improve the output properties of these microorganisms. Strains which are resistant to antimetabolites, such as, for example, the lysine analogue S-(2-aminoethyl)-cysteine or the valine analogue 2-thiazolyl-alanine, or are auxotrophic for metabolites of regulatory importance and produce L-amino acids are obtained in this manner.

Methods of the recombinant DNA technique have also been employed for some years for improving the strain of Corynebacterium glutamicum strains which produce L-amino acids, by amplifying individual amino acid biosynthesis genes and investigating the effect on the L-amino acid production.

### OBJECT OF THE INVENTION

The inventors had the object of providing new principles for improved processes for the fermentative preparation of L-amino acids with coryneform bacteria.

### DESCRIPTION OF THE INVENTION

Where L-amino acids or amino acids are mentioned in the following, this means one or more amino acid, including their salts, chosen from the group consisting of L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine. L-Lysine and L-valine are particularly preferred.

When L-lysine or lysine are mentioned in the following, not only the bases but also the salts, such as e.g. lysine monohydrochloride or lysine sulfate, are meant by this.

When L-valine or valine are mentioned in the following, the salts, such as e.g. valine monohydrochloride or valine sulfate are also meant by this.

The invention provides a process for the fermentative preparation of L-amino acids using coryneform bacteria in which at least the nucleotide sequence which codes for quinolinic acid synthetase A (quinolinate synthetase A) (nadA gene) and/or the nucleotide sequence which codes for nicotinate nucleotide pyrophosphorylase (nadC gene) is or are attenuated, in particular eliminated or expressed at a low level.

This invention also provides a process for the fermentative preparation of L-amino acids, in which the following steps are carried out:

a) fermentation of the L-amino acid-producing coryneform bacteria in which at least the nucleotide sequence which codes for quinolinic acid synthetase (nadA) and/or the nucleotide sequence which codes for [sic] (nadC) is or are attenuated, in particular eliminated or expressed at a low level;

b) concentration of the L-amino acids in the medium or in the cells of the bacteria; and

c) isolation of the L-amino acids produced.

The strains employed preferably already produce L-amino acids, in particular L-valine or L-lysine, before attenuation of the nadA and/or nadC gene.

Preferred embodiments are to be found in the claims.

The term “attenuation” in this connection describes the reduction or elimination of the intracellular activity of one or more enzymes (proteins) in a microorganism which are coded by the corresponding DNA, for example by using a weak promoter or using a gene or allele which codes for a corresponding enzyme with a low activity or inactivates the corresponding gene or enzyme (protein), and optionally combining these measures.

The microorganisms to which the present invention relates can prepare amino acids from glucose, sucrose, lactose, fructose, maltose, molasses, starch, cellulose or from glycerol and ethanol. They can be representatives of coryneform bacteria, in particular of the genus Corynebacterium. Of the genus Corynebacterium, there may be mentioned in particular the species Corynebacterium glutamicum, which is known among experts for its ability to produce L-amino acids.

Suitable strains of the genus Corynebacterium, in particular of the species Corynebacterium glutamicum, are in particular the known wild-type strains

Corynebacterium glutamicum ATCC13032

Corynebacterium acetoglutamicum ATCC15806

Corynebacterium acetoacidophilum ATCC13870

Corynebacterium melassecola ATCC17965

Corynebacterium thermoaminogenes FERM BP-1539

Brevibacterium flavum ATCC14067

Brevibacterium lactofermentum ATCC13869 and

Brevibacterium divaricatum ATCC14020

and L-amino acid-producing mutants or strains prepared therefrom

such as, for example, the L-lysine-producing strains

Corynebacterium glutamicum FERM-P 1709

Brevibacterium flavum FERM-P 1708

Brevibacterium lactofermentum FERM-P 1712

Corynebacterium glutamicum FERM-P 6463

Corynebacterium glutamicum FERM-P 6464 and

Corynebacterium glutamicum DSM 5714

or such as, for example, the valine-producing strains

Corynebacterium glutamicum DSM 12455

Corynebacterium glutamicum FERM-P 9325

Brevibacterium flavum FERM-P 512

Brevibacterium lactofermentum FERM-P 1845

Brevibacterium lactofermentum FERM-P 9324 and

Brevibacterium lactofermentum FERM-BP 1763.

It has been found that coryneform bacteria produce L-amino acids in an improved manner after attenuation of the nadA and/or nadC gene.

The sequences of the nadA and nadC genes are shown in SEQ ID No. 1 and 3 and can be used according to the invention. The amino acid sequences of the associated gene products are shown in SEQ ID No. 2 and 4. Alleles of the nadA and/or nadC gene which result from the degeneracy of the genetic code or due to “sense mutations” of neutral function can furthermore be used.

To achieve an attenuation, either the expression of the nadA and/or nadC gene or the catalytic properties of the gene products can be reduced or eliminated. The two measures are optionally combined.

The gene expression can be reduced by suitable culturing or by genetic modification (mutation) of the signal structures of gene expression. Signal structures of gene expression are, for example, repressor genes, activator genes, operators, promoters, attenuators, ribosome binding sites, the start codon and terminators. The expert can find information on this e.g. in the patent application WO 96/15246, in Boyd and Murphy (Journal of Bacteriology 170: 5949 (1988)), in Voskuil and Chambliss (Nucleic Acids Research 26: 3548 (1998), in Jensen and Hammer (Biotechnology and Bioengineering 58: 191 (1998)), in Pátek et al. (Microbiology 142: 1297 (1999)) and in known textbooks of genetics and molecular biology, such as e.g. the textbook by Knippers (“Molekulare Genetik [Molecular Genetics]”, 6th edition, Georg Thieme Verlag, Stuttgart, Germany, 1995) or that by Winnacker (“Gene und Klone [Genes and Clones]”, VCH Verlagsgesellschaft, Weinheim, Germany, 1990).

Mutations which lead to a change or reduction in the catalytic properties of enzyme proteins are known from the prior art; examples which may be mentioned are the works by Qiu and Goodman (Journal of Biological Chemistry 272: 8611-8617 (1997)), Sugimoto et al. (Bioscience Biotechnology and Biochemistry 61: 1760-1762 (1997)) and Möckel (“Die Threonindehydratase aus Corynebacterium glutamicum: Aufhebung der allosterischen Regulation und Struktur des Enzyms [Threonine dehydratase from Corynebacterium glutamicum: Cancelling the allosteric regulation and structure of the enzyme]”, Reports from the Julich Research Centre, Jül-2906, ISSN09442952, Jülich, Germany, 1994). Summarizing descriptions can be found in known textbooks of genetics and molecular biology, such as e.g. that by Hagemann (“Allgemeine Genetik [General Genetics]”, Gustav Fischer Verlag, Stuttgart, 1986).

Possible mutations are transitions, transversions, insertions and deletions. Depending on the effect of the amino acid exchange on the enzyme activity, “missense mutations” or “nonsense mutations” are referred to. Insertions or deletions of at least one base pair in a gene lead to “frame shift mutations”, as a consequence of which incorrect amino acids are incorporated or translation is interrupted prematurely. Deletions of several codons typically lead to a complete loss of the enzyme activity. Instructions on generation of such mutations are prior art and can be found in known textbooks of genetics and molecular biology, such as e.g. the textbook by Knippers (“Molekulare Genetik [Molecular Genetics]”, 6th edition, Georg Thieme Verlag, Stuttgart, Germany, 1995), that by Winnacker (“Gene und Klone [Genes and Clones]”, VCH Verlagsgesellschaft, Weinheim, Germany, 1990) or that by Hagemann (“Allgemeine Genetik [General Genetics]”, Gustav Fischer Verlag, Stuttgart, 1986).

A common method of mutating genes of C. glutamicum is the method of “gene disruption” and “gene replacement” described by Schwarzer and Puhler (Bio/Technology 9, 84-87 (1991)).

In the method of gene disruption a central part of the coding region of the gene of interest is cloned in a plasmid vector which can replicate in a host (typically E. coli), but not in C. glutamicum. Possible vectors are, for example, pSUP301 (Simon et al., Bio/Technology 1, 784-791 (1983)), pK18mob or pK19mob (Schäfer et al., Gene 145, 69-73 (1994)), pK18mobsacB or pK19mobsacB (Jäger et al., Journal of Bacteriology 174: 5462-65 (1992)), pGEM-T (Promega corporation, Madison, Wis., U.S.A.), pCR2.1-TOPO (Shuman (1994). Journal of Biological Chemistry 269:32678-84; U.S. Pat. No. 5,487,993), pCR®Blunt (Invitrogen, Groningen, Holland; Bernard et al., Journal of Molecular Biology, 234: 534-541 (1993)) or pEM1 (Schrumpf et al, 1991, Journal of Bacteriology 173:4510-4516). The plasmid vector which contains the central part of the coding region of the gene is then transferred into the desired strain of C. glutamicum by conjugation or transformation. The method of conjugation is described, for example, by Schäfer et al. (Applied and Environmental Microbiology 60, 756-759 (1994)). Methods for transformation are described, for example, by Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). After homologous recombination by means of a “cross-over” event, the coding region of the gene in question is interrupted by the vector sequence and two incomplete alleles are obtained, one lacking the 3′ end and one lacking the 5′ end. This method has been used, for example, by Fitzpatrick et al. (Applied Microbiology and Biotechnology 42, 575-580 (1994)) to eliminate the recA gene of C. glutamicum.

The plasmid pCR2.1nadAint, with the aid of which the process of disruption of the nadA gene can be carried out, is shown by way of example in FIG. 1. Plasmid pCR2.1nadAint contains a central part of the nadA gene, which is called the nadAint fragment and is shown in SEQ ID No. 5.

The plasmid pCR2.1nadCint, with the aid of which the process of disruption of the nadC gene can be carried out, is furthermore shown in FIG. 2. Plasmid pCR2.1nadCint contains a central part of the nadC gene, which is called the nadCint fragment and is shown in SEQ ID No. 6.

In the method of “gene replacement”, a mutation, such as e.g. a deletion, insertion or base exchange, is established in vitro in the gene of interest. The allele prepared is in turn cloned in a vector which is not replicative for C. glutamicum and this is then transferred into the desired host of C. glutamicum by transformation or conjugation. After homologous recombination by means of a first “cross-over” event which effects integration and a suitable second “cross-over” event which effects excision in the target gene or in the target sequence, the incorporation of the mutation or of the allele is achieved. This method was used, for example, by Peters-Wendisch et al. (Microbiology 144, 915- 927(1998)) to eliminate the pyc gene of C. glutamicum by a deletion.

A deletion, insertion or a base exchange can be incorporated into the nadA and/or nadC gene in this manner.

In addition, it may be advantageous for the production of L-amino acids to enhance, in particular over-express, one or more enzymes of the particular biosynthesis pathway, of glycolysis, of anaplerosis, of the citric acid cycle, of the pentose phosphate cycle, of amino acid export and optionally regulatory proteins, in addition to the attenuation of the nadA and/or nadC gene.

The term “enhancement” or “enhance” in this connection describes the increase in the intracellular activity of one or more enzymes or proteins in a microorganism which are coded by the corresponding DNA, for example by increasing the number of copies of the gene or genes, using a potent promoter or a gene which codes for a corresponding enzyme or protein with a high activity, and optionally combining these measures.

Thus, for the preparation of L-lysine, in addition to attenuation of the nadA and/or nadC gene, one or more of the genes chosen from the group consisting of

the lysC gene which codes for a feed-back resistant aspartate kinase (Accession No. P26512),

the dapA gene which codes for dihydrodipicolinate synthase (EP-B 0 197 335),

the gap gene which codes for glyceraldehyde 3-phosphate dehydrogenase (Eikmanns (1992). Journal of Bacteriology 174:6076-6086),

at the same time the pyc gene which codes for pyruvate carboxylase (DE-A-198 31 609),

the mqo gene which codes for malate:quinone oxidoreductase (Molenaar et al., European Journal of Biochemistry 254, 395-403 (1998)),

the zwf gene which codes for glucose 6-phosphate dehydrogenase (JP-A-09224661),

at the same time the lysE gene which codes for lysine export (DE-A-195 48 222),

the zwa1 gene which codes for the Zwa1 protein (DE: 19959328.0, DSM 13115)

the tpi gene which codes for triose phosphate isomerase (Eikmanns (1992), Journal of Bacteriology 174:6076-6086), and

the pgk gene which codes for 3-phosphoglycerate kinase (Eikmanns (1992), Journal of Bacteriology 174:6076-6086),

can be enhanced, in particular over-expressed.

Thus, for the preparation of L-valine, in addition to attenuation of the nadA and/or nadC gene, one or more of the genes chosen from the group consisting of

the lysC gene which codes for a feed-back resistant aspartate kinase (Accession No. P26512),

the hom gene which codes for homoserine dehydrogenase (EP-A 0131171),

the hom(Fbr) allele which codes for a feed-back resistant homoserine dehydrogenase (Reinscheid et al. (1991) Journal of Bacteriology 173: 3228-3230),

the ilvA gene which codes for threonine dehydratase (Möckel et al., Journal of Bacteriology (1992) 8065-8072)), or the ilvA(Fbr) allele which codes for a feed-back resistant threonine dehydratase (Möckel et al., (1994) Molecular Microbiology 13: 833-842),

the ilvBN gene which codes for acetohydroxy-acid synthase (EP-B 0356739),

the ilvD gene which codes for dihydroxy-acid dehydratase (Sahm and Eggeling (1999) Applied and Environmental Microbiology 65: 1973-1979),

the zwf gene which codes for glucose 6-phosphate dehydrogenase (JP-A-09224661),

at the same time the brnF- and/or brnE genes which code for valine export (DE: 19951708.8), and

the zwa1 gene which codes for the Zwa1 protein (DE: 19959328.0, DSM 13115)

can be enhanced, in particular over-expressed.

It may furthermore be advantageous for the production of amino acids, in particular L-lysine and L-valine, in addition to the attenuation of the nadA and/or nadC gene, at the same time for one or more of the genes chosen from the group consisting of

the pck gene which codes for phosphoenol pyruvate carboxykinase (DE 199 50 409.1, DSM 13047),

the pgi gene which codes for glucose 6-phosphate isomerase (U.S. patent application Ser. No. 09/396,478, DSM 12969),

the poxB gene which codes for pyruvate oxidase (DE:1995 1975.7, DSM 13114),

the zwa2 gene which codes for the Zwa2 protein (DE: 19959327.2, DSM 13113)

to be attenuated, in particular for the expression thereof to be reduced.

Finally, in addition to attenuation of the nadA and/or nadC gene it may be advantageous for the production of amino acids to eliminate undesirable side reactions (Nakayama: “Breeding of Amino Acid Producing Micro-organisms”, in: Overproduction of Microbial Products, Krumphanzl, Sikyta, Vanek (eds.), Academic Press, London, UK, 1982).

The invention also provides the microorganisms prepared according to the invention, and these can be cultured continuously or discontinuously in the batch process (batch culture) or in the fed batch (feed process) or repeated fed batch process (repetitive feed process) for the purpose of production of L-amino acids. A summary of known culture methods is described in the textbook by Chmiel (Bioprozesstechnik 1. Einführung in die Bioverfahrenstechnik [Bioprocess Technology 1. Introduction to Bioprocess Technology (Gustav Fischer Verlag, Stuttgart, 1991)) or in the textbook by Storhas (Bioreaktoren und periphere Einrichtungen [Bioreactors and Peripheral Equipment] (Vieweg Verlag, Braunschweig/Wiesbaden, 1994)).

The culture medium to be used must meet the requirements of the particular strains in a suitable manner. Descriptions of culture media for various microorganisms are contained in the handbook “Manual of Methods for General Bacteriology” of the American Society for Bacteriology (Washington D.C., U.S.A., 1981).

Sugars and carbohydrates, such as e.g. glucose, sucrose, lactose, fructose, maltose, molasses, starch and cellulose, oils and fats, such as e.g. soya oil, sunflower oil, groundnut oil and coconut fat, fatty acids, such as e.g. palmitic acid, stearic acid and linoleic acid, alcohols, such as e.g. glycerol and ethanol, and organic acids, such as e.g. acetic acid, can be used as the source of carbon. These substance can be used individually or as a mixture.

Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture.

Phosphoric acid, potassium dihydrogen phosphate or dipotassium hydrogen phosphate or the corresponding sodium-containing salts can be used as the source of phosphorus. The culture medium must furthermore comprise salts of metals, such as e.g. magnesium sulfate or iron sulfate, which are necessary for growth. Finally, essential growth substances, such as amino acids and vitamins, can be employed in addition to the abovementioned substances. Suitable precursors can moreover be added to the culture medium. The starting substances mentioned can be added to the culture in the form of a single batch, or can be fed in during the culture in a suitable manner.

Basic compounds, such as sodium hydroxide, potassium hydroxide, ammonia or aqueous ammonia, or acid compounds, such as phosphoric acid or sulfuric acid, can be employed in a suitable manner to control the pH of the culture. Antifoams, such as e.g. fatty acid polyglycol esters, can be employed to control the development of foam. Suitable substances having a selective action, such as e.g. antibiotics, can be added to the medium to maintain the stability of plasmids. To maintain aerobic conditions, oxygen or oxygen-containing gas mixtures, such as e.g. air, are introduced into the culture. The temperature of the culture is usually 20° C. to 45° C., and preferably 25° C. to 40° C. Culturing is continued until a maximum of the desired product has formed. This target is usually reached within 10 hours to 160 hours.

Methods for the determination of L-amino acids are known from the prior art. The analysis can thus be carried out as described by Spackman et al. (Analytical Chemistry, 30, (1958), 1190) by anion exchange chromatography with subsequent ninhydrin derivatization, or it can be carried out by reversed phase HPLC, for example as described by Lindroth et al. (Analytical Chemistry (1979) 51: 1167-1174).

The present invention is explained in more detail in the following with the aid of embodiment examples.

### EXAMPLE 1

Preparation of a genomic cosmid gene library from Corynebacterium glutamicum ATCC 13032

Chromosomal DNA from Corynebacterium glutamicum ATCC 13032 is isolated as described by Tauch et al. (1995, Plasmid 33:168-179) and partly cleaved with the restriction enzyme Sau3AI (Amersham Pharmacia, Freiburg, Germany, Product Description Sau3AI, Code no. 27-0913-02). The DNA fragments are dephosphorylated with shrimp alkaline phosphatase (Roche Diagnostics GmbH, Mannheim, Germany, Product Description SAP, Code no. 1758250). The DNA of the cosmid vector SuperCos1 (Wahl et al. (1987) Proceedings of the National Academy of Sciences USA 84:2160-2164), obtained from Stratagene (La Jolla, U.S.A., Product Description SuperCos1 Cosmid Vector Kit, Code no. 251301) is cleaved with the restriction enzyme XbaI (Amersham Pharmacia, Freiburg, Germany, Product Description XbaI, Code no. 27-0948-02) and likewise dephosphorylated with shrimp alkaline phosphatase.

The cosmid DNA is then cleaved with the restriction enzyme BamHI (Amersham Pharmacia, Freiburg, Germany, Product Description BamHI, Code no. 27-0868-04). The cosmid DNA treated in this manner is mixed with the treated ATCC13032 DNA and the batch is treated with T4 DNA ligase (Amersham Pharmacia, Freiburg, Germany, Product Description T4-DNA-Ligase, Code no. 27-0870-04). The ligation mixture is then packed in phages with the aid of Gigapack II XL Packing Extract (Stratagene, La Jolla, U.S.A., Product Description Gigapack II XL Packing Extract, Code no. 200217).

For infection of the E. coli strain NM554 (Raleigh et al. 1988, Nucleic Acid Research 16:1563-1575) the cells are taken up in 10 mM MgSO₄ and mixed with an aliquot of the phage suspension. The infection and titering of the cosmid library are carried out as described by Sambrook et al. (1989, Molecular Cloning: A laboratory Manual, Cold Spring Harbor), the cells being plated out on LB agar (Lennox, 1955, Virology, 1:190) with 100 mg/l ampicillin. After incubation overnight at 37° C., recombinant individual clones are selected.

### EXAMPLE 2

Isolation and sequencing of the nadA gene

The cosmid DNA of an individual colony is isolated with the Qiaprep Spin Miniprep Kit (Product No. 27106, Qiagen, Hilden, Germany) in accordance with the manufacturer's instructions and partly cleaved with the restriction enzyme Sau3AI (Amersham Pharmacia, Freiburg, Germany, Product Description Sau3AI, Product No. 27-0913-02). The DNA fragments are dephosphorylated with shrimp alkaline phosphatase (Roche Diagnostics GmbH, Mannheim, Germany, Product Description SAP, Product No. 1758250). After separation by gel electrophoresis, the cosmid fragments in the size range of 1500 to 2000 bp are isolated with the QiaExII Gel Extraction Kit (Product No. 20021, Qiagen, Hilden, Germany).

The DNA of the sequencing vector pZero-1, obtained from Invitrogen (Groningen, Holland, Product Description Zero Background Cloning Kit, Product No. K2500-01), is cleaved with the restriction enzyme BamHI (Amersham Pharmacia, Freiburg, Germany, Product Description BamHI, Product No. 27-0868-04). The ligation of the cosmid fragments in the sequencing vector pZero-1 is carried out as described by Sambrook et al. (1989, Molecular Cloning: A laboratory Manual, Cold Spring Harbor), the DNA mixture being incubated overnight with T4 ligase (Pharmacia Biotech, Freiburg, Germany). This ligation mixture is then electroporated (Tauch et al. 1994, FEMS Microbiol Letters, 123:343-7) into the E. coli strain DH5αMCR (Grant, 1990, Proceedings of the National Academy of Sciences U.S.A., 87:4645-4649) and plated out on LB agar (Lennox, 1955, Virology, 1:190) with 50 mg/l zeocin.

The plasmid preparation of the recombinant clones is carried out with a Biorobot 9600 (Product No. 900200, Qiagen, Hilden, Germany). The sequencing is carried out by the dideoxy chain termination method of Sanger et al. (1977, Proceedings of the National Academy of Sciences U.S.A., 74:5463-5467) with modifications according to Zimmermann et al. (1990, Nucleic Acids Research, 18:1067). The “RR dRhodamin Terminator Cycle Sequencing Kit” from PE Applied Biosystems (Product No. 403044, Weiterstadt, Germany) was used. The separation by gel electrophoresis and analysis of the sequencing reaction are carried out in a “Rotiphoresis NF Acrylamide/Bisacrylamide” Gel (29:1) (Product No. A124.1, Roth, Karlsruhe, Germany) with the “ABI Prism 377” sequencer from PE Applied Biosystems (Weiterstadt, Germany).

The raw sequence data obtained are then processed using the Staden program package (1986, Nucleic Acids Research, 14:217-231) version 97-0. The individual sequences of the pZero1 derivatives are assembled to a continuous contig. The computer-assisted coding region analysis is prepared with the XNIP program (Staden, 1986, Nucleic Acids Research 14:217-231).

The resulting nucleotide sequence is shown in SEQ ID No. 1. Analysis of the nucleotide sequence shows an open reading frame of 1287 base pairs, which is called the nadA gene. The nadA gene codes for a protein of 428 amino acids.

### EXAMPLE 3

Isolation and sequencing of the nadC gene

The isolation and sequencing of the nadC gene is carried out as described in example 2.

The resulting nucleotide sequence is shown in SEQ ID No. 3. Analysis shows an open reading frame of 840 base pairs, which is called the nadC gene. The nadC gene codes for a polypeptide of 279 amino acids.

### EXAMPLE 4

Preparation of an integration vector for integration mutagenesis of the nadA gene

From the strain ATCC 13032, chromosomal DNA is isolated by the method of Eikmanns et al. (Microbiology 140: 1817 -1828 (1994)). On the basis of the sequence of the nadA gene known for C. glutamicum from example 2, the following oligonucleotides are chosen as primers for the polymerase chain reaction:

nadAint1 (shown in SEQ ID No. 7)

5′ AAG CGA TTG TGT TCT GCG GT 3′

nadAint2 (shown in SEQ ID No. 8)

5′ TCG AGG CAG AAG ATG GTG TG 3′

The primers shown are synthesized by ARK, Scientific GmbH Biosystems (Darmstadt, Germany) and the PCR reaction is carried out by the standard PCR method of Innis et al. (PCR protocols. A guide to methods and applications, 1990, Academic Press) with Pwo-Polymerase from Boehringer. With the aid of the polymerase chain reaction, an DNA fragment 780 bp in size is isolated, this being an internal fragment of the nadA gene. It is shown in SEQ ID No. 5.

The amplified DNA fragment is ligated with the TOPO TA Cloning Kit from Invitrogen Corporation (Carlsbad, Calif., U.S.A.; Catalogue Number K4500-01) in the vector pCR2.1-TOPO (Mead at [sic] al. (1991) Bio/Technology 9:657-663). The E. coli strain DH5αmcr is then electroporated with the ligation batch (Hanahan, In: DNA cloning. A practical approach. Vol.I. IRL-Press, Oxford, Washington D.C., U.S.A., 1985). Selection for plasmid-carrying cells is made by plating out the transformation batch on LB agar (Sambrook et al., Molecular cloning: a laboratory manual. 2^{nd} Ed. Cold Spring Harbor Laboratory Press, Cold Spring Harbor, N.Y., 1989), which is supplemented with 25 mg/l kanamycin. Plasmid DNA is isolated from a transformant with the aid of the QIAprep Spin Miniprep Kit from Qiagen and checked by restriction with the restriction enzyme EcoRI and subsequent agarose gel electrophoresis (0.8%). The plasmid is called pCR2.1nadAint. It is shown in FIG. 1.

### EXAMPLE 5

Preparation of an Integration Vector for Integration Mutagenesis of the NadC Gene

As described in example 3, chromosomal DNA is isolated from the strain ATCC 13032 by the method of Eikmanns et al. (Microbiology 140: 1817-1828 (1994)). On the basis of the sequence of the nadC gene known for C. glutamicum from example 3, the following oligonucleotides are chosen as primers for the polymerase chain reaction:

nadCint1 (shown in SEQ ID No. 9)

5′ AGC TGA GCG CCA AGG TTG TT 3′

nadCint2 (shown in SEQ ID No. 10)

5′ CGA TGA GCT GAT CAA TGG TG 3′

The primers shown are synthesized by ARK, Scientific GmbH Biosystems (Darmstadt, Germany) and the PCR reaction is carried out by the standard PCR method of Innis et al. (PCR protocols. A guide to methods and applications, 1990, Academic Press) with Pwo-Polymerase from Boehringer. With the aid of the polymerase chain reaction, a DNA fragment 582 bp in size is isolated, this being an internal fragment of the nadC gene. It is shown in SEQ ID No. 6.

The amplified DNA fragment is ligated with the TOPO TA Cloning Kit from Invitrogen Corporation (Carlsbad, Calif., U.S.A.; Catalogue Number K4500-01) in the vector pCR2.1-TOPO (Mead at [sic] al. (1991) Bio/Technology 9:657-663). The E. coli strain DH5αmcr is then electroporated with the ligation batch (Hanahan, In: DNA cloning. A practical approach. Vol. I. IRL-Press, Oxford, Washington D.C., U.S.A., 1985). Selection for plasmid-carrying cells is made by plating out the transformation batch on LB agar (Sambrook et al., Molecular cloning: a laboratory manual. 2^{nd} Ed. Cold Spring Harbor Laboratory Press, Cold Spring Harbor, N.Y., 1989), which is supplemented with 25 mg/l kanamycin. Plasmid DNA is isolated from a transformant with the aid of the QIAprep Spin Miniprep Kit from Qiagen and checked by restriction with the restriction enzyme EcoRI and subsequent agarose gel electrophoresis (0.8%). The plasmid is called pCR2.1nadCint. It is shown in FIG. 2.

### EXAMPLE 6

Integration mutagenesis of the nadA gene in the C. glutamicum strain DM678

The vector pCR2.lnadAint mentioned in example 4 is electroporated by the electroporation method of Tauch et al.(FEMS Microbiological Letters, 123:343-347 (1994)) in Corynebacterium glutamicum DM678.

The Corynebacterium glutamicum strain DM678 was prepared by multiple mutagenesis and selection from strain ATCC13032. This strain is methionine-sensitive. A pure culture of the strain DM678 was deposited on Jun. 15, 1999 at the Deutsche Sammlung für Mikroorganismen und Zellkulturen (DSM=German Collection of Microorganisms and Cell Cultures, Braunschweig, Germany) as DSM12866.

The vector pCR2.1nadAint cannot replicate independently in DM678 and is retained in the cell only if it has integrated into the chromosome of DM678. Selection of clones with pCR2.1nadAint integrated into the chromosome is carried out by plating out the electroporation batch on LB agar (Sambrook et al., Molecular cloning: A Laboratory Manual, 2^{nd} Ed., Cold Spring Harbor Laboratory Press, Cold Spring Harbor, N.Y.), which is supplemented with 15 mg/l kanamycin.

For detection of the integration, the nadAint fragment is labelled with the Dig hybridization kit from Boehringer by the method of “The DIG System Users Guide for Filter Hybridization” of Boehringer Mannheim GmbH (Mannheim, Germany, 1993). Chromosomal DNA of a potential integrant is isolated by the method of Eikmanns et al. (Microbiology 140: 1817-1828 (1994)) and in each case cleaved with the restriction enzymes SalI, SacI and HindIII. The fragments formed are separated by agarose gel electrophoresis and hybridized at 68° C. with the Dig hybrization [sic] kit from Boehringer. The plasmid pCR2.1nadAint mentioned in example 4 has been inserted into the chromosome of DM678 within the chromosomal nadA gene. The strain is called DM678::pCR2.inadAint.

### EXAMPLE 7

Preparation of L-valine with the aid of the Corynebacterium glutamicum strain DM678::pCR2.1nadAint

The C. glutamicum strain DM678::pCR2.1nadAint obtained in example 6 was first incubated on an agar plate with the corresponding antibiotic (brain-heart agar with 25 mg/l kanamycin) for 24 hours at 33° C. Starting from this agar plate, a preculture was seeded (40 ml medium in a 500 ml conical flask). The medium SK65 was used as the medium for the preculture.

Medium SK65

| | | |
|---|---|---|
| CSL (corn steep liquor) | 25 | g/l |
| Glucose | 23 | g/l |
| Peptone from casein | 20 | g/l |
| Peptone from meat | 20 | g/l |
| Ammonium sulfate | 8 | g/l |
| Urea | 3 | g/l |
| Salts: | | |
| KH₂PO₄ | 2.0 | g/l |
| MgSO₄ * 7 H₂O | 0.5 | g/l |
| FeSO₄ * 7 H₂O | 10 | mg/l |
| CuSO₄ | 1.0 | mg/ml |
| ZnSO₄ | 10 | mg/l |
| Thiamine * HCl (sterile-filtered) | 0.5 | mg/l |
| Biotin (sterile-filtered) | 0.2 | mg/l |
| CaCO₃ | 1.6 | g/l |

The preculture was incubated for 20 hours at 33° C. at 170 rpm on a shaking machine.

2.5 g of this preculture were inoculated into 831 g of fermentation medium M1-457. The culturing fermentation was carried out in 2 l stirred reactor fermenters from B.Braun (BBI, Germany, Melsungen, Biostat MD model). The medium M1-457 contained the constituents listed in table 1. After being filled with the medium, the fermenter was sterilized for 30 minutes at 121° C. Only starch hydrolysate, Ca pantothenate and thiamine were added in a sterile-filtered form after the autoclaving. The culture was cultured at a temperature of 32° C., an aeration of 1 l/min, a minimum stirrer speed of 800 rpm and a pH of 7.0 and an oxygen partial pressure of 20% air saturation until the sugar initially introduced had been consumed. The culture was then cultured for a further 38 hours at a temperature of 34° C., an oxygen partial pressure of 20% air saturation and a pH value of pH 7.0 until an OD660 of 30.5 was reached. During this period, 273.6 g medium M2-242 with a glucose concentration of 506.3 g/l, an ammonium sulfate concentration of 35.7 g/l and a KH₂PO₄ concentration of 1.342 g/l were fed in.

Thereafter, the optical density (OD) was determined with a digital photometer of the type LP1W from Dr. Bruno Lange GmbH (Berlin, Germany) at a measurement wavelength of 660 nm and the concentration of L-valine formed was determined by means of ASA.

In the end sample of the fermentation, an L-valine concentration of 160 mg/l was found after the end of the fermentation. No L-valine could be detected in a parallel fermentation with the control strain C. glutamicum DM 678.

Composition of medium M1-457

| | Concentration | |
|---|---|---|
| Component | (per kg) | |
| Starch hydrolysate | 28.9 | g |
| (87.6%) | | |
| (NH₄)₂SO₄ | 55.50 | g |
| CSL | 19.57 | g |
| MgSO₄ · 7H₂O | 0.678 | g |
| FeSO₄ · 7H₂O | 50 | mg |
| ZnSO₄ · H₂O | 50 | mg |
| KH₂PO₄ | 1.1 | g |
| Citric acid H₂O | 1.165 | g |
| CuSO₄ · 5H₂O | 1.5 | mg |
| Thiamine | 0.3 | mg |
| D(+)Biotin | 0.1 | mg |
| Nicotinic acid | 2.8 | mg |
| Structol | 0.6 | g |

### DESCRIPTION OF THE FIGURES

FIG. 1: Map of the plasmid pCR2.1nadAint

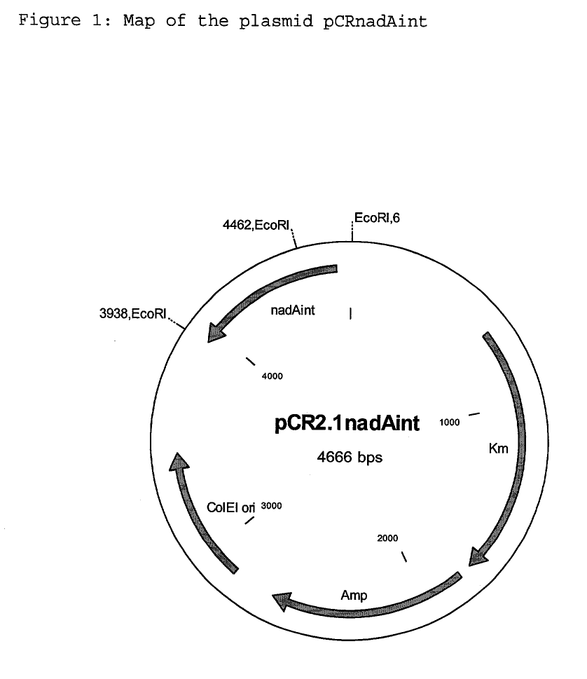

The abbreviations and designations used have the following meaning. The base pair numbers stated are approximate values obtained in the context of reproducibility of measurements.

| | |
|---|---|
| Kam: | Resistance gene for kanamycin |
| Amp: | Resistance gene for ampicillin |
| ColEI ori: | Replication origin ColEI from |
| | Escherichia coli |
| EcoRI: | Cleavage site of the restriction enzyme EcoRI |
| nadAint: | Internal fragment of the nadA gene |

FIG. 2: Map of the plasmid pCR2.1nadCint

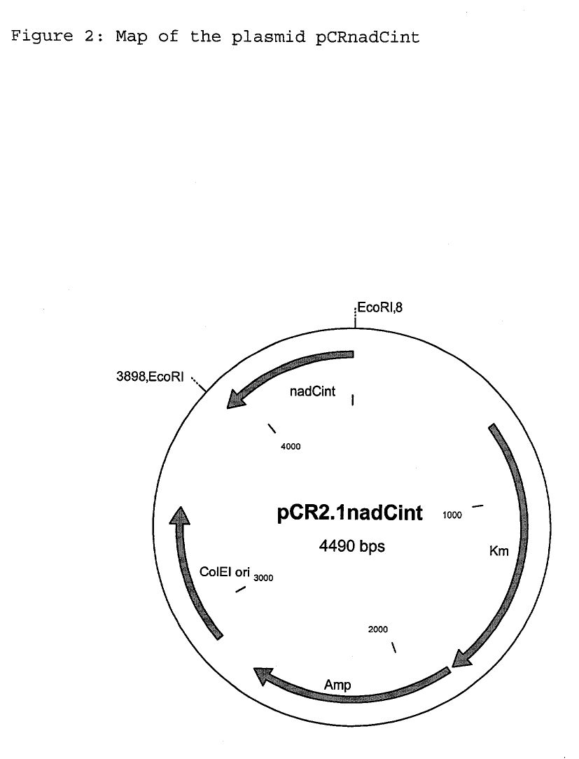

The abbreviations and designations used have the following meaning. The base pair numbers stated are approximate values obtained in the context of reproducibility of measurements.

| | |
|---|---|
| Kam: | Resistance gene for kanamycin |
| Amp: | Resistance gene for ampicillin |
| ColEI ori: | Replication origin ColEI from |
| | Escherichia coli |
| EcoRI: | Cleavage site of the restriction enzyme EcoRI |
| nadCint: | Internal fragment of the nadC gene |

1. A process for the fermentative preparation of L-amino acids, in particular L-lysine and L-valine, which comprises carrying out the following steps,
a) fermentation of the coryneform bacteria which produce the desired L-amino acid and in which at least the nadA and/or nadC gene is or are attenuated,
b) concentration of the desired product in the medium or in the cells of the bacteria and
c) isolation of the L-amino acid.

2. A process as claimed in claim 1, wherein bacteria in which further genes of the biosynthesis pathway of the desired L-amino acid are additionally enhanced are employed.

3. A process as claimed in claim 1, wherein bacteria in which the metabolic pathways which reduce the formation of the desired L-amino acid are at least partly eliminated are employed.

4. A process as claimed in claim 1, wherein the expression of the polynucleotide(s) which code(s) for the nadA and/or nadC gene is reduced.

5. A process as claimed in claim 1, which comprises reducing the regulatory/catalytic properties of the polypeptide (enzyme protein) for which the polynucleotide nadA and/or nadC code(s).

6. A process as claimed in claim 1, wherein for the preparation of L-lysine, coryneform microorganisms in which at the same time one or more of the genes chosen from the group consisting of
6.1 the lysC gene which codes for a feed-back resistant aspartate kinase,
6.2 the dapA gene which codes for dihydrodipicolinate synthase,
6.3 the gap gene which codes for glyceraldehyde 3-phosphate dehydrogenase,
6.4 the pyc gene which codes for pyruvate carboxylase,
6.5 the mqo gene which codes for malate:quinone oxidoreductase,
6.6 the zwf gene which codes for glucose 6-phosphate dehydrogenase,
6.7 at the same time the lysE gene which codes for lysine export,
6.8 the zwa1 gene which codes for the Zwa1 protein
6.9 the tpi gene which codes for triose phosphate isomerase, and
6.10 the pgk gene which codes for 3-phosphoglycerate kinase,
is or are enhanced, in particular over-expressed, are fermented.

7. A process as claimed in claim 1, wherein for the preparation of L-valine, coryneform microorganisms in which at the same time one or more of the genes chosen from the group consisting of formatting-bolding
7.1 the lysC gene which codes for a feed-back resistant aspartate kinase,
7.2 the hom gene which codes for homoserine dehydrogenase
7.3 the hom(Fbr) allele which codes for a feed-back resistant homoserine dehydrogenase,
7.4 the ilvA gene which codes for threonine dehydratase, or the ilvA(Fbr) allele which codes for a feed-back resistant threonine dehydratase,
7.5 the ilvBN gene which codes for acetohydroxy-acid synthase,
7.6 the ilvD gene which codes for dihydroxy-acid dehydratase,
7.7 the zwf gene which codes for glucose 6-phosphate dehydrogenase,
7.8 at the same time the brnF- and/or brnE genes which code for valine export, and
7.9 the zwa1 gene which codes for the Zwa1 protein
is or are enhanced, in particular over-expressed, are fermented.

8. A process as claimed in claim 1, wherein for the preparation of L-amino acids, coryneform microorganisms in which at the same time one or more of the genes chosen from the group consisting of
8.1 the pck gene which codes for phosphoenol pyruvate carboxykinase,
8.2 the pgi gene which codes for glucose 6-phosphate isomerase
8.3 the poxB gene which codes for pyruvate oxidase, or
8.4 the zwa2 gene which codes for the Zwa2 protein
is or are attenuated are fermented.

9. A process as claimed in one or more of the preceding claims, wherein microorganisms of the species Corynebacterium glutamicum are employed.
